(19) United States
(12) Patent Application Publication
(10) Pub. No.: US 2019/0024148 A1
(43) Pub. Date: Jan. 24, 2019

(54) NUCLEIC ACID AMPLIFICATION BIOSENSOR FOR USE IN ROLLING CIRCLE AMPLIFICATION (RCA)

(71) Applicant: McMaster University, Hamilton (CA)

(72) Inventors: Yingfu Li, Dundas (CA); John Brennan, Dundas (CA); Meng Liu, Dundas (CA)

(73) Assignee: McMaster University, Hamilton, ON (CA)

(21) Appl. No.: 16/060,633

(22) PCT Filed: Dec. 12, 2016
(86) PCT No.: PCT/CA2016/051458

§ 371 Date: Jun. 8, 2018

Related U.S. Application Data
(60) Provisional application No. 62/266,225, filed on Dec. 11, 2015.

(51) Int. Cl. C12Q 1/6825; C12Q 1/6844

(52) U.S. Cl. CPC C12Q 1/6825; C12Q 1/6844; C12Q 2565/625; C12Q 2531/125; C12Q 2565/519; C12Q 2527/125

(57) ABSTRACT

The present application describes a biosensor for detecting target nucleic acid. The biosensor's mode of operation is based on binding of the target nucleic acid to another nucleic acid sequence and a circular template which triggers rolling circle amplification and detection of the amplified product as the indicator of the presence of the target nucleic acid. The biosensor is immobilized on a solid support, such as paper.

The present application claims the benefit of priority from U.S. provisional patent application No. 62/266,225, filed Dec. 11, 2015, the contents of which are herein incorporated by reference.

### FIELD

The present application relates to a biosensor for detecting analytes, various kits and methods of use thereof. In particular, the biosensor's mode of operation is based on binding of nucleic acid analytes to a nucleic acid sequence which triggers rolling circle amplification and detection of the amplified product as the indicator of the presence of the analytes.

### BACKGROUND

There is currently a great need for developing rapid and effective point-of-care (POC) diagnostics that can improve patient care in resource-limited settings.^{[1]} Paper-based POC diagnostic devices provide a platform for portable, low-cost, low-volume, disposable, and simple sensors,^{[2]} which can be developed using inkjet printing,^{[3]} wax printing^{[4]} or screen printing technology,^{[5]} making them amenable to automated fabrication or even on-site production in areas with limited resources.^{[6]}

One major challenge of molecular amplification technology is sensitive target detection that is fast, inexpensive, simple and convenient. Recently, isothermal nucleic acid amplification techniques have been widely investigated as a method to facilitate target or signal amplification in molecular biology and bioanalysis without the use of thermocycling devices.^{[7]}

### SUMMARY

Herein, rolling circle amplification (RCA), a simple and efficient isothermal enzymatic DNA replication process,^{[9]} that is performed in a microzone plate fabricated, for example, on a paper solid support (paper-based RCA) using wax printing is described, along with a theoretical basis for understanding the nucleic acid amplification reaction on paper, and in particular, the intriguing finding that RCA efficiency on paper is enhanced relative to solution. An “all-in-one” paper-based amplification system, in which all required reagents for amplification and detection are integrated via printing into the sensor, providing improved functionality and ease of use, is also demonstrated. Thus the combination of isothermal nucleic acid amplification techniques with paper-based POC diagnostics will improve the robustness and sensitivity of these devices.

In one aspect, the present application includes a nucleic acid sensor probe for detection of target nucleic acid comprising:
(a) a capture oligonucleotide that comprises a nucleic acid sequence that is complementary to a first nucleic acid sequence of the target nucleic acid;
(b) a solid support immobilizing the capture oligonucleotide; and
(c) reagents for performing rolling-circle amplification (RCA) of the target nucleic acid.

In some embodiments the reagents for performing RCA of the target nucleic acid are comprised in a stabilized composition. For example the reagents are encapsulated in a stabilizing matrix or are freeze dried.

The present application also includes a method of determining the presence of target nucleic acid in a sample comprising:
a) exposing a nucleic acid sensor probe of the application to the sample;
b) performing rolling-circle amplification (RCA) on the nucleic acid sensor probe under conditions to generate single-stranded nucleic acid molecules; and
c) detecting the single-stranded nucleic acid molecules generated in b),
   wherein the detection of the single-stranded nucleic molecules in c) indicates the presence of the target nucleic acid in the sample.

In another aspect, the present disclosure describes a nucleic acid sensor probe for detection of target DNA. In one embodiment, nucleic acid sensor probe is comprised of a DNA oligonucleotide that is complementary to part of the target DNA, a solid support for immobilizing the DNA oligonucleotide and encapsulated reagents for performing rolling-circle amplification of the target DNA. In one embodiment, the nucleic acid sensor probe contains a DNA oligonucleotide that is biotinylated and bound with streptavidin. In one embodiment, the nucleic acid sensor probe has a solid support that is made of paper. In one embodiment, the nucleic acid sensor probe contains reagents for performing rolling-circle amplification encapsulated with pullulan.

In another aspect, the present disclosure provides methods for determining the presence of target DNA in a sample. In one embodiment, the method comprises exposing the sensor probe to a DNA oligonucleotide that is complementary to part of the circular template of the target DNA in a sample; generating single stranded DNA molecules by rolling-circle amplification in the presence of the DNA oligonucleotide and the circular template; detecting the single stranded DNA molecules generated by rolling-circle amplification; and wherein the detection of the single stranded DNA molecule generated by rolling-circle amplification indicates the presence of the target in the sample. In one embodiment, detection of single stranded DNA molecules generated by rolling-circle amplification is indicated by colour, or incorporation of radioactive or fluorophore molecules

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DRAWINGS

The embodiments of the application will now be described in greater detail with reference to the attached drawings in which:

FIG. 1 shows an image of an exemplary paper plate made of 96 microzones printed with fluorescent DNA-streptavidin DNA conjugates.

FIG. 2 illustrates the RCA with paper-bound primer in an exemplary embodiment of the application, including different exemplary methods for detection of RCA products (RP): (A) through incorporation of radioactive tracer into DNA chain; (B) fluorescent assay using a fluorophore-labeled DNA oligonucleotide; (C) colorimetric assay using AuNP-DNA conjugates; (D) colorimetric assay mediated by a peroxidase-mimicking DNAzyme through the oxidation of 3,3′,5,5′-tetramethylbenzidine (TMB) in the presence of hemin and H₂O₂.

FIG. 3 shows a colorimetric assay with the RCA mixtures taken from the exemplary paper-based RCA in FIG. 2D. After the RCA reaction on paper, the mixture was transferred to a test-tube using a pipette. Then 1 μL of 40 mM H₂O₂ and 1 μL of 20 mM TMB (3,3′,5,5′-tetramethylbenzidine) was added to initiate the colorimetric reaction. The image was captured by a digital camera ˜1 minute after all components were added.

FIG. 4 shows the dPAGE analysis of digested RCA products produced by FastDigestEcoRV under different digestion times in exemplary embodiments of the application.

FIG. 5 shows the determination of RCA efficiency of exemplary embodiments of the application. (A) dPAGE analysis of digested RP obtained at varying RCA times with free TP2 (F-TP2) and paper-bound TP2 (P-TP2). Top band: digested RCA monomer (60 nt). Bottom band: DNA loading control (50 nt). FR: ratio of fluorescence intensity of the 60-nt and 50-nt DNA bands. ARU: average repeating units of RP from a given circular template. (B) Average repeating units (ARU) of RP vs. RCA time for F-TP2 and P-TP2. (C) Schematic illustration showing that the immobilization of DNA primers on paper in exemplary embodiments of the application substantially increases the localized concentrations of the primer-circular template complex.

FIG. 6 shows the efficiency comparison of exemplary paper-based RCA and solution-based RCA. (A) RCA with varying concentrations of TP2. (B) RCA with 0.25 μM TP3. The concentration of circular template was 5 nM. Top band: digested RCA monomer (60 nt). Bottom band: DNA loading control (50 nt). FR: ratio of fluorescence intensity of the 60-nt and 50-nt DNA bands. ARU: average repeating units in RP.

FIG. 7 illustrates (A) a schematic diagram of the preparation of an exemplary all-in-one bioactive paper sensor using pullulan. (B) Typical images of exemplary pullulan tablets. (C) Dose-response curves for HCV-1 DNA detection with an exemplary bioactive paper sensor. Inset: a photograph for visual detection on a chip array at HCV-1 at various concentrations. Two rows represent two repeats. (D) Evaluation of the long-term stability of the exemplary paper sensors stored at 4° C. and room temperature (RT).

FIG. 8 shows RCA reactions of the exemplary bioactive paper sensors in the presence of CDT3, HCV-1 or both. Each reaction was performed for 40 min at RT in 15 μL of 1× RCA buffer containing the indicated components of 5 nM CDT3 and 0.1 nM HCV-1 before adding1 μL of 40 mM H₂O₂ and 1 μL of 20 mM TMB to initiate the colorimetric reaction.

FIG. 9 shows the selectivity of exemplary bioactive paper sensors for DNA detection. The reaction conditions were identical to FIG. 8.

FIG. 10 shows the RCA efficiency with and without exemplary pullulan encapsulation.

FIG. 11 shows exemplary quantification of miR-21 contents in total small RNA (<200 nt) of (A) MCF-7 and (B) MCF-10A cells by standard addition methods.

FIG. 12 shows exemplary absolute quantification of miR-21 in MCF-7 and MCF-10A cell lines using Quantitative Reverse Transcription-PCR (qRT-PCR). qRT-PCR reactions were performed using a gScript™ microRNA cDNA Synthesis Kit according to manufacturer's instructions. The 50 μL qRT-PCR solution contained 15 ng total small RNA sample, or miR-21 with desired amounts of RNA. Melt-curve analysis of PCR product indicated the specificity of the reaction. cDNA representing 15 ng of total small RNA (<200 nt) was assayed for miR-21. The C_{T} values for each sample are shown in Table 2.

### DETAILED DESCRIPTION

I. Definitions

Unless otherwise indicated, the definitions and embodiments described in this and other sections are intended to be applicable to all embodiments and aspects of the present application herein described for which they are suitable as would be understood by a person skilled in the art.

As used herein in this specification and the appended claims, the singular forms “a”, “an” and “the” include plural references unless the content clearly dictates otherwise. Thus for example, a composition containing “an analyte” includes one such analyte or a mixture of two or more analytes.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

As used in this application and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The term “and/or” as used herein means that the listed items are present, or used, individually or in combination. In effect, this term means that “at least one of” or “one or more” of the listed items is used or present.

The term “suitable” as used herein means that the selection of the particular compound or conditions would depend on the specific manipulation to be performed, but the selection would be well within the skill of a person trained in the art.

The term “solid support” as used herein refers to any solid support to which one or more nucleic acid molecules can be printed.

The term “paper” or “paper-based material” as used herein refers to a commodity of thin material produced by the amalgamation of fibers, typically plant fibers composed of cellulose, which are subsequently held together by hydrogen bonding.

As used herein, the term “immobilized” or synonyms thereof in reference to the capture oligonucleotide, means that the movement of the oligonucleotide molecules of the biosensor is restricted.

The term “encapsulated” as used herein means that the referenced agents are either physically or chemically located within a matrix to that movement in and out of the matrix is restricted.

The term “nucleic acid” as used herein refers to a biopolymer made from monomers of nucleotides. Each nucleotide has three components: a 5-carbon sugar, a phosphate group and a nitrogenous base. If the sugar is deoxyribose, the biopolymenr is DNA (deoxyribonucleic acid). If the sugar is ribose, the biopolymer is RNA (ribonucleic acid). When all three components are combined, they form a nucleotide.

The term “target nucleic acid” as used herein means any nucleic acid molecule (DNA or RNA) which one would like to sense or detect using a biosensor of the present application

The term “sample(s)” as used herein refers to any material that one wishes to assay for the presence of the target oligonucleotide using the biosensor of the application.

The term “reporter molecules for detection” as used herein refers to one or more molecules that are used to detect the presence of target oligonucleotide.

The term “detection system” as used herein refers to any means that produces a signal that is detectable, for example, using colorimetric, fluorescent, electrochemical and/or radioimaging methods, when the target nucleic acid is present and RCA takes place.

The term “oligonucleotide” as used herein refers to short single stranded DNA or RNA oligomers that are either synthetic or found in nature. Oligonucleotides are characterized by the sequence of nucleotide residues that make up the entire molecule. The length of the oligonucleotide is usually denoted by “-mer”. For example, an oligonucleotide of six nucleotides (nt) is a hexamer, while one of 25 nt would usually be called a “25-mer”. Oligonucleotides readily bind, in a sequence-specific manner, to their respective complementary oligonucleotides, DNA or RNA, to form duplexes.

The term “rolling circle amplification” or “RCA” as used herein refers to a unidirectional nucleic acid replication that can rapidly synthesize multiple copies of circular molecules of DNA or RNA. In an embodiment, rolling circle amplification is an isothermal enzymatic process where a short DNA or RNA primer is amplified to form a long single-stranded DNA or RNA using a circular DNA template and an appropriate DNA or RNA polymerase.

The term “printing” as used herein refers to the placement of a substance on a solid support using a mechanical device that prints the substance onto the solid support.

The term “exonucleolytic trimming” or “exonucleolytic digestion” as used herein refers to the cleaving of nucleotides one at a time from the end (exo) of a polynucleotide chain by a nucleic acid exonuclease.

II. Biosensors of the Application

The present application includes a nucleic acid sensor probe for detection of target nucleic acid comprising:
a) a capture oligonucleotide that comprises a nucleic acid sequence that is complementary to a first nucleic acid sequence of the target nucleic acid;
b) a solid support immobilizing the capture oligonucleotide; and
c) reagents for performing rolling-circle amplification (RCA) of the target nucleic acid.

In some embodiments, the capture oligonucleotide is immobilized by binding to a membrane and the membrane-bound oligonucleotide is printed on to the solid support. In some embodiments, the capture oligonucleotide is biotinylated and bound with streptavidin on a membrane. In some embodiments, the membrane is a nitrocellulose membrane. A person skilled in the art would appreciate that there are many methods for immobilizing oligonucleotides to solid supports and the present application is not limited to any particular method.

In some embodiments, the solid support comprises a substantially planar surface. In some embodiments, the solid support is made from paper, glass, plastic, polymers, metals, ceramics, alloys or composites. In some embodiments, the solid support is made from paper or a paper-based material.

In an embodiment, the “paper” or “paper-based material” is an amalgamation of plant fibers composed of cellulose. In an embodiment, the plant fibers are from wood pulp from pulpwood trees. In an embodiment, the plant fibers are from pulpwood, cotton, hemp, linen or rice, or a mixture thereof. While the fibers used are usually natural in origin, it is an embodiment that a wide variety of synthetic fibers, such as polypropylene and polyethylene, are incorporated into the paper as a way of imparting desirable physical properties.

In some embodiments the reagents for performing RCA are comprised in a stabilized composition. In some embodiments the stabilized composition allows the sensor probe to be stored for extended periods of time, for example at temperatures of about −20° C. to about 25° C., for about 1 day to about 1 year. In some embodiments, the sensor probe is stored at temperatures of about 0° C. to about 25° C., for about 1 day to about 1 month. In some embodiments, the sensor probe is stored at temperatures of about 4° C. to about 25° C., for about 1 day to about 15 days.

In some embodiments, the stabilized composition is stabilized by freeze drying or lyophilization. In some embodiments the stabilized composition is stabilized by encapsulation into a water soluble solid polymer matrix. U.S. Pat. No. 7,604,807 describes the reversible preservation of biological samples in compositions comprising natural polymers such as pullullan or acacia gum. PCT patent application no. WO 2015/066819 describes methods for performing chemical reactions in which two or more of the reagents for the chemical reactions are stabilized using water soluble polymers. The contents of both of these documents as it relates to stabilizing biomolecules in water soluble polymer matrixes, is incorporated herein by reference.

In some embodiments, the water soluble solid polymeric matrix is a polysaccharide. In some embodiments, the water soluble solid polymeric matrix is comprised of pullulan. Pullulan is a natural polysaccharide produced by the fungus Aureobasidium pullulans. It readily dissolves in water but resolidifies into films upon drying.

In some embodiments, the water soluble solid polymeric matrix dissolves upon addition of a sample to the sensor probe. Dissolution of the polymeric matrix results in the release of the RCA reagents.

In some embodiments of the application, each of the reagents for performing RCA are in separate solid polymeric matrixes.

In some embodiments the reagents for performing RCA comprise one or more of a circular template comprising a single stranded nucleic acid sequence that is complementary to a second nucleic acid sequence of the target nucleic acid, a nucleic acid polymerase having exonuclease activity, RCA reaction buffer and nucleoside triphosphates (NTPs). In some embodiments the reagents for performing RCA comprise a circular template comprising a nucleic acid sequence that is complementary to a second nucleic acid sequence of the target nucleic acid, a nucleic acid polymerase having exonuclease activity, RCA reaction buffer and nucleoside triphosphates (NTPs).

In some embodiments, the NTPs are ATP, GTP, UTP and CTP. In some embodiments, NTPs are deoxynucleoside triphosphates (dNTPs) selected from dATP, dGTP, dUTP and dCTP. In some embodiments, the NTPs are radiolabeled.

In some embodiments the nucleic acid polymerase is a DNA polymerase having 3′ to 5′ exonuclease activity or an RNA polymerase having 3′ to 5′ exonuclease activity. In some embodiments, the nucleic acid polymerase is a DNA polymerase. In some embodiments, the nucleic acid polymerase is phi29 DNA polymerase (ϕ29DP).

In some embodiments, a complex is formed between the immobilized capture oligonucleotide, the circular template and target nucleic acid and, upon formation of this complex, RCA proceeds to produce a detectable RCA product (RP).

In some embodiments, the stabilized composition comprising reagents for performing RCA is printed on top of the immobilized capture oligonucleotide and the sensor probe allowed to dry for storage prior to use.

In some embodiments, the printing of the capture oligonucleotide and/or the stabilized composition comprising reagents for performing RCA is performed using a non-contact microarray printer. In some embodiments, the printing of the capture oligonucleotide and/or the stabilized composition comprising reagents for performing RCA is performed using inkjet printing, wax printing or screen printing technology, suitably wax printing. A person skilled in the art would appreciate that there are many methods for applying the capture oligonucleotide and/or the stabilized composition comprising reagents for performing RCA to the solid support, including many printing methods, and the present application is not limited to any particular method.

In some embodiments, the sensor probe of the application further comprises one or more reporter molecules for detection of the RP. In some embodiments, the one or more reporter molecules for detection of the target oligonucleotide comprise a detection system selected from a fluorescent system, a colorimetric system, a radiolabeled system and an electrochemical system. In some embodiments, the one or more reporter molecules are incorporated in the reagents for performing RCA and/or in the capture oligonucleotide.

In some embodiments, a radiolabeled dNTP, such as an [α-³²P]dNTP is included in the reagents for performing RCA so that the RCA product (RP) becomes radioactive, and therefore detectable. In some embodiments, a fluorophore-labeled capture oligonucleotide is used that can hybridize with the RP to produce a detectable fluorescence signal. In some emboduments, gold nanoparticles (AuNPs) functionalized with an oligonucleotide that is complementary to the capture oligonucleotide is used to produce a detectable colorimetric signal. In some embodiments, a modified circular template that produces a special RP containing repetitive units of a peroxidase-mimicking DNAzyme, PW17, generating a detectable colorimetric signal is used.

In some embodiments, the presence of the RCA product is monitored using an electrophoresis system and the presence of the target nucleic acid is confirmed by detection of a specific molecular weight band. The process of preparing the sample, preparing the gel and subsequent visualization techniques of the electrophoresis system are well known in the prior art.

In some embodiments, the capture oligonucleotides and target nucleic acid are selected from DNA molecules and/or RNA molecules. In some the capture oligonucleotides and target nucleic acid are DNA molecules. In some embodiments, the capture oligonucleotides and target nucleic acid are RNA molecules.

The target nucleic acid is any nucleic acid molecule that has been generated by any means that one wishes to detect. In some embodiments, the target nucleic acid is either isolated from a natural source or is synthetic. In some embodiments the target nucleic acid is a nucleic acid from a microorganism. In some embodiments, the microorganism is a bacterium, virus or fungi. In some embodiments, the microorganism is a bacterium. In some embodiments, the microorganism is a virus. In some embodiments, the target nucleic is released from a source using, for example, DNA enzymes, RNA enzymes or aptamers. In some embodiments the target nucleic acid is an antisense sequence of a nucleic acid molecule. In some embodiments, the antisense sequence of a nucleic acid molecule is an antisense sequence of a viral nucleic acid sequence or an antisense sequence of a bacterial nucleic acid sequence.

In some embodiments, the target nucleic acid molecule is the product of a reaction or the action of other molecules, such as enzymes, in systems that one wishes to detect. In this embodiment, the presence of the target nucleic acid indirectly indicates the presence of the system via detecting its reaction products. In some embodiments, the system is in a microorganism.

In some embodiments, the target nucleic acid is a nucleic acid from a biological sample, the presence of which is indicative of or associated with a disease, disorder or condition. In some embodiments, the target nucleic acid is from a sample comprising cells and tissue that are assayed for the presence of specific RNA or DNA associated with a disease, disorder or condition. In some embodiments, the disease, disorder or condition is cancer and the biololgical sample comprises cancer cells or tumor cells.

A person skilled in the art would appreciate that the sensor probe of the application contains a plurality of target oligonucleotides immobilized on the solid support. In some embodiments, the plurality of target oligonucleotides are immobilized in a microzone plate fabricated on a paper or paper-based material. In some embodiments, each of the microzones comprising the plurality of target oligonucleotides has printed thereon the stabilized reagents for performing rolling-circle amplification (RCA) of the target nucleic acid. In operation, addition of a sample suspected of comprising the target nucleic acid is added to each microzone, releasing the reagents for performing RCA, and, if the target is present in the sample, RCA products are detected.

The sample may be from any source, for example, any biological (for example human or animal medical samples), environmental (for example water or soil) or natural (for example plants) source, or from any manufactured or synthetic source (for example food or drinks). The sample is one that comprises or is suspected of comprising one or more target nucleic acids. In some embodiments, the sample is treated to release the target nucleic acid prior to application to the sensor probe of the application.

The present application also includes a nucleic acid sensor probe for detection of target DNA comprised of:
a) a DNA oligonucleotide that is complementary to part of the target DNA
b) a solid support for immobilizing the DNA oligonucleotide
c) encapsulated reagents for performing rolling-circle amplification of the target DNA.

In some embodiments, the nucleic acid sensors of the application are comprised in a kit. Accordingly, the present application also includes a target oligonucleotide detection kit comprising a nucleic acid sensor probe of the application. In some embodiments, the kit further comprises reagents for performing an assay using the nucleic acid sensor probe of the application. In some embodiments, the kit further comprises instructions for using the nucleic acid sensor probe in the assay and any controls needed to perform the assay.

III. Methods of the Application

The present application includes a method of determining the presence of target nucleic acid in a sample comprising:
a) exposing the nucleic acid sensor probe of the application to the sample;
b) performing rolling-circle amplification (RCA) on the nucleic acid sensor probe under conditions to generate single-stranded nucleic acid molecules; and
c) detecting single-stranded nucleic acid molecules generated in b),
   wherein the detection of the single-stranded nucleic molecule in c) indicates the presence of the target nucleic acid in the sample.

The present application also includes a method of determining the presence of target DNA in a sample comprising:
a) exposing the sensor probe with a DNA oligonucleotide that is complementary to part of the circular template of the target DNA in a sample
b) generating single stranded DNA molecules by rolling-circle amplification in the presence of the DNA oligonucleotide and the circular template
c) detecting the single stranded DNA molecules generated in b)
wherein the detection of the single stranded DNA molecule in c) indicates the presence of the target in the sample.

In some embodiments, the detection of single-stranded nucleic acid molecules generated by RCA is indicated by a colorimetric system, a fluorescence system, a radiolabeling system or an electrochemical system as described above.

In some embodiments, the detection of single stranded DNA molecules generated by rolling circle amplification is indicated by colour, electrochemistry or incorporation of radioactive or fluorescent molecules

The present application also includes assay methods that utilize the biosensor of the present application. In an embodiment, the assay is a method of detecting target nucleic acid in a sample, wherein the sample comprises or is suspected of comprising the target nucleic acid molecule, the method comprising contacting the sample with the biosensor of the application and monitoring for a presence of a nucleic acid product from the RCA template wherein the presence of the nucleic acid product from the RCA template indicates the presence of the target nucleic acid molecule in the sample.

In some embodiments, the detection of the target nucleic acid is performed by monitoring for the presence of a RCA nucleic acid product. In this embodiment, the nucleic acid product being formed possesses a detectable signal (for e.g., fluorescence, molecular weight as described above) that is distinct from the signal of any of the starting reagents.

In some embodiments, the presence of the RCA product comprises a detection system. In an embodiment, the detection system is selected from a fluorescent system, a colorimetric system, an electrophoresis system and an electrochemical system.

In some embodiments, the presence of the RCA product is monitored using an electrophoresis system and the presence of the target nucleic acid is confirmed by detection of a single molecular weight band. The process of preparing the sample, preparing the gel and subsequent visualization techniques of the electrophoresis system are well known in the prior art.

In some embodiments, the presence of the RCA product is monitored using a fluorescent system and the presence of the analyte is confirmed by detection of a fluorescent signal. In some embodiments, the fluorescent system comprises a fluorescent reporter molecule that monitors the progression of the nucleic acid product amplification. Depending on the mode of signal generation, the fluorescent reporter molecule is either a fluorogenically labelled oligonucleotide, referred to as a probe, or a fluorogenic nucleotide-binding dye.

The sample may be from any source, for example, any biological (for example human or animal medical samples), environmental (for example water or soil) or natural (for example plants) source, or from any manufactured or synthetic source (for example food or drinks). The sample is one that comprises or is suspected of comprising one or more target nucleic acids. In some embodiments, the sample is treated to concentrate the target nucleic acids prior to application to the biosensor of the application.

In some embodiments, the sample is sample is from a microorganism. In some embodiments, the microorganism is a bacterium, virus or fungi. In some embodiments, the microorganism is a bacterium. In some embodiments, the microorganism is a virus. In some embodiments, the target nucleic is released from the sample using, for example, DNA enzymes, RNA enzymes or aptamers.

In some embodiments, the sample is a biological sample and the presence of the target nucleic acid in the sample is indicative of or associated with a disease, disorder or condition. In some embodiments, the biological sample comprises cells or tissues that are assayed for the presence of specific RNA or DNA associated with a disease, disorder or condition. In some embodiments, the disease, disorder or condition is cancer and the biololgical sample comprises cancer cells or tumor cells.

### EXAMPLES

The following non-limiting examples are illustrative of the present application:

### Example 1

### Development of Biosensors

### Materials

### Oligonucleotides and Other Materials

All DNA oligonucleotides (Table 1) were obtained from Integrated DNA Technologies (IDT), and purified by standard 10% denaturing (8 M urea) polyacrylamide gel electrophoresis (dPAGE) or high-performance liquid chromatography (HPLC). T4 polynucleotide kinase (PNK), T4 DNA ligase and phi29 DNA polymerase (ϕ29DP) were purchased from MBI Fermentas (Burlington, Canada). α-[³²P]dGTP was purchased from PerkinElmer. SYBR Gold (10,000× concentrated stock in DMSO) was purchased from Life Technologies (Burlington, ON, Canada). Pullulan (molecular weight of ˜200,000 Daltons) was obtained from Polysciences Inc. Nitrocellulose membranes (HF180) were acquired from Millipore. All other chemicals were purchased from Sigma-Aldrich (Oakville, Canada) and used without further purification.

### Instruments

The autoradiogram and fluorescent images of gels were obtained using a Typhoon 9200 variable mode imager (GE healthcare) and analyzed using Image Quant software (Molecular Dynamics). Paper micro-well plates were printed using a Xerox ColorQube 8570N solid wax printer.

### Preparation of Circular DNA Templates

Circular DNA templates were synthesized from 5′-phosphorylated linear DNA oligonucleotides through template-assisted ligation using T4 DNA ligase. The protocol for phosphorylation of DNA was as follows: a total of 200 pmol LDT1 or LDT2 was first mixed with 10 U (U: unit) PNK and 2 mM ATP in 50 μL of 1× PNK buffer A (final concentration: 50 mM Tris-HCl, pH 7.6 at 25° C., 10 mM MgCl₂, 5 mM DTT, 0.1 mM spermidine). The mixture was incubated at 37° C. for 40 min, followed by heating at 90° C. for 5 min. Then 250 pmol CD1 was added, heated at 90° C. for 5 min, cooled down and left at room temperature for 20 min. To the above mixture were added 15 μL of 10× T4 DNA ligase buffer (400 mM Tris-HCl, 100 mM MgCl₂, 100 mM DTT, 5 mM ATP, pH 7.8 at 25° C.) and 10 U T4 DNA ligase, and the resultant mixture (total 150 μL) was incubated at RT for 2 h before heating at 90° C. for 5 min to deactivate the ligase. The ligated circular DNA products were concentrated by standard ethanol precipitation and purified by 10% dPAGE.

### Preparation of AuNPs-DP2 Conjugates

AuNPs with an average diameter of 13 nm (with a final concentration of 22 nM) were prepared by using the citrate reduction method. AuNPs were conjugated with DP2 to have approximately 100 DNA sequences per particle. Briefly, 2.2 nmol thiolated DP2 was activated with 1.5 μL of 10 mM Tris(2-carboxyethyl)phosphine (TCEP) at RT for 1 h. Then 1 mL of AuNPs was added and gently shaken overnight. Tris-acetate buffer (pH 8.2) was added to the above solution to produce a final concentration of 20 mM. Following the addition of 100 mM NaCl, AuNP-DP1 conjugates were aged in salt for 24 h. Excess reagents were removed by centrifuging at 14,000 rpm for 20 min. The red precipitate was washed twice and dispersed in 25 mM PBS containing 150 mM NaCl, 0.02% Tween-20 and 10% (w/v) sucrose.

### Preparation of Bioactive Paper

To facilitate the immobilization of DNA primers for RCA on paper, streptavidin was used to bind the biotinylated TP1. Briefly, 1 nmol of TP1 and 30 μL of 2 mg/mL streptavidin were added to 300 μL of PBS buffer (25 mM containing 150 mM NaCl, 5 mM MgCl₂, pH 7.6). After incubating at RT for 2 h, free TP1 was removed by centrifugation through a 30 K membrane (NANOSEP OMEGA, Pall Incorporation) at 5,000 g for 10 min. The as-prepared streptavidin-biotin DNA conjugates were washed twice with PBS buffer and collected. Then a desired volume of the above solution was printed onto each test zone with Scienion SciFlex Arrayer S5 Non-Contact Microarray Printer and allowed to dry at RT. After immersion in PBS buffer (containing 10% BSA) for 20 min and washing twice, the obtained bioactive paper was dried at RT and stored at 4° C. in a desiccant container.

### Methods

### RCA Reaction on Paper

In a typical experiment, 1.5 μL of 10× RCA reaction buffer (330 mM Tris acetate, 100 mM magnesium acetate, 660 mM potassium acetate, 1% (v/v) Tween-20, 10 mM DTT, pH 7.9), 5 U of ϕ29DP, 1 μL of 10 mM dNTPs and 1 μL of 1 μM CDT1 or CDT2 were added (total volume: 15 μL) to the test zones. The reaction was allowed to proceed at RT for 40 min. For the radioactive assay, 0.5 μL of [α-³²P]dGTP was added before the RCA reaction. Then the bioactive paper was immersed in PBS buffer and washed twice. For the fluorescent assay and AuNP-based colorimetric assay, 1 μL of 100 μM DP1 or freshly prepared AuNPs-DP2 conjugates was introduced to each well and allowed to hybridize to the RCA products for 20 min at RT. The patterned paper was then washed by immersing into PBS buffer and dried under nitrogen before being scanned. For the DNAzyme-based colorimetric assay, 1 μL of 100 μM hemin (dissolved in DMSO) was added before the RCA reaction. Then 1 μL of 40 mM H₂O₂ and 1 μL of 20 mM TMB (3,3′,5,5′-tetramethylbenzidine) was added to initiate the colorimetric reaction after the RCA reaction, and a photograph was taken using a digital camera within ˜1 minute after all the reaction components were added.

### Comparison of RCA Efficiency for Solution-based RCA and Paper-based RCA

RCA reaction. For solution-based RCA, 1 μL of 0.1 μM CDT1 was first mixed with 1 μL of 5 μM TP2 (or TP3), 1 μL of 5 μM DC1, 2 μL of 10× RCA buffer, 1 μL of 10 mM dNTPs and 5 U of ϕ29DP. This mixture was incubated at 30° C. for 5, 10, 20, 30 and 60 min before heating at 65° C. for 15 min to deactivate the polymerase. For paper-based RCA, 1 μL of 0.1 μM CDT1, 1 μL of 5 μM TP2 (or TP3), 2 μL of 10× RCA reaction buffer, 1 μL of 10 mM dNTPs and 5 U of ϕ29DP were added to five parallel test zones coated with streptavidin-DC1 conjugates. The reaction was allowed to proceed in a humidity chamber at 30° C. for 5, 10, 20, 30 and 60 min before adding 20 μL of 8 M urea to elute the RCA products from the paper. For RCA reaction using TP3, the RCA reaction time was 20 min. For comparison of the RCA efficiency under different concentrations of TP2, TP2 with final concentrations of 0.025 μM, 0.25 μM, 2.5 μM and 25 μM were tested at the RCA reaction time of 20 min. The obtained RCA products were concentrated by standard ethanol precipitation.

Restriction digestion. A 4 μL aliquot of the above RCA products was mixed with 3 μL of 100 μM DT1 and heated at 90° C. for 5 min before cooled to RT and left for 20 min. This was followed by the addition of 1 μL of 10× Fast digestion buffer and 2 μL of FastDigestEcoRV. The reaction mixture was then incubated at 37° C. for 24 h.

Analysis of monomeric RCA products. 10 μL of the digestion products was mixed with 3 μL of 800 nM internal control DNA (ICD) and 10 μL of 2× denaturing gel loading buffer. The mixture was then run on a 10% dPAGE gel and stained with 1× SYBR Gold at 4° C. for 15 min before scanning.

Calculation of FR and ARU. The fluorescence intensity of the monomeric DNA band (F₆₀ₙₜ) and the ICD1 band (F₅₀ₙₜ) from each digestion mixture was estimated using Image Quant software and used to calculate the fluorescence ratio (FR) using: FR=F₆₀ₙₜ/F₅₁ₙₜ. From this, we can calculate the total amount of the monomeric DNA using: n₆₀ₙₜ=FR×2.4 pmol×5. Note that 5 was the volume correction factor, which was calculated from 20/4 (4 μL of the RCA reaction mixture from 20 μL was used for the digestion reaction). Since the concentration of CDT1 was the limiting factor for the RCA reaction, the average repeating units (ARU) of the RCA product can be estimated using: ARU=(FR×2.4 pmol×5)/0.1 pmol.

### Preparation of Pullulan-Encapsulated RCA Reagents on Paper

1 μL of 1 μM CDT3, 1 μL of 10 mM dNTPs, 1 μL of 100 μM hemin and 5 U of ϕ29DP were first mixed with 20 μL of 10% (w/v) pullulan. The mixtures were printed onto the DC2-coated paper using a Biodot XYZ3060 automated dispensing unit as described elsewhere.^{[1]} After drying at RT, the fully bioactive paper was stored at 4° C. or RT in a desiccant container. To test its long-term stability, 20 μL of 1× RCA buffer containing 100 nM HCV-1 DNA was added to dissolve the pullulan film on paper. The RCA reaction was allowed to proceed in a humidity chamber at 30° C. for 30 min before adding 1 μL of 40 mM H₂O₂ and 1 μL of 20 mM TMB to initiate the colorimetric reaction. The colorimetric result was recorded within ˜1 minute by a digital camera.

### Theoretical Calculation of the Localized Concentration

If we assume that a solution contains 10 nM of DNA primers and circular templates, the volume of a sphere containing one DNA molecule would be:

V=1 L/(10×10⁻⁹ mol×6.023×10²³ mol⁻¹)=0.17 fL

and thus the calculated sphere radius is:

r=(3V/4π)^{1/3}=(3×0.17×10⁹ nm³/4π)^{1/3}=340 nm.

If the complementary DNA molecules are immobilized on paper, each one will be confined in a hemisphere with the radius of 10 nm. Thus the volume of the hemisphere would be:

V=(1/2)×4πr³/3=(1/2)×4π×(10 nm)³/3=2.1 zL.

Then the localized concentration of these DNA molecules was estimated to be:

C=2 n/NV=2 mol/(6.023×10²³×4.2 zL)=800 μM.

Cell Culture and miRNA Extraction

The adherent breast cancer cell line MCF-7 was cultured in α-MEM media (GIBCO) supplemented with 10% fetal bovine serum (Invitrogen). MCF-10A (mammary epithelial cell line) was cultured in D-MEM medium supplemented with 5% (v/v) horse serum, 10 μg/mL human insulin, 10 ng/mL epidermal growth factor, 500 ng/mL hydrocortisone and 10 μM isoproterenol. These cells were cultured at 37° C. in a humidified atmosphere containing 5% CO₂. RNAs (<200 nt) were extracted and purified using the E.Z.N.A® miRNA Kit according to the manufacturer's protocol including 1) cell lysis, 2) organic extraction, 3) large RNA removal and 4) miRNA purification. The RNA quantity was determined by measuring optical density at 260 nm using the NanoVue™ Plus spectrophotometer.

Quantification of miR-21 in Cells by Paper-based Assay

1 μL of 1 μM CDT4, 1 μL of 10 mM dNTPs, 1 μL of 100 μM hemin and 5 U of ϕ29DP were first mixed with 20 μL of 10% pullulan. The mixtures were printed onto the DC3-coated paper using a Biodot XYZ3060 automated dispensing unit.^{[1]} The total small RNA of MCF-7 and MCF-10A was adjusted to 0.2 μg/μL and 0.8 μg/μL with nuclease-free water, respectively. 1 μL of the total RNA sample was employed for measurement using the standard addition method with synthetic miR-21 as the standard. The RCA reaction was initiated by the addition of 20 μL of 1× RCA buffer containing the spiked miR-21 and incubated in a humidity chamber at 30° C. for 30 min before adding 1 μL of 40 mM H₂O₂ and 1 μL of 20 mM TMB for the colorimetric reaction.

### Results

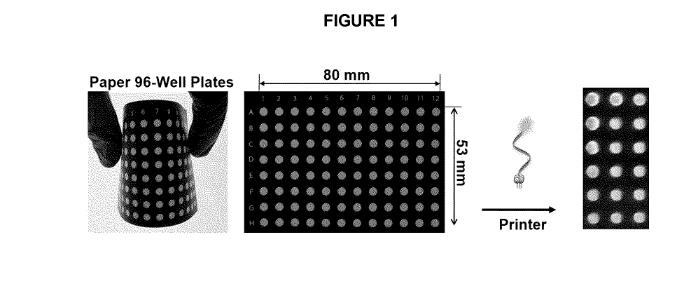

To make a paper device for RCA, a preformed 5′-biotinylated DNA-streptavidin conjugate was printed onto a nitrocellulose membrane surface, which is known to have high affinity for protein binding. The sequence of the printed DNA molecule was designed to be complementary to part of a circular DNA template (CDT) and thus can act as a primer for RCA. To demonstrate that the proposed strategy allows efficient printing of DNA primers onto a paper surface, the following experiment was performed. First, the wax-printing technique was used to produce a 96-microzone paper plate, with the diameter of each test zone being 4 mm. A fluorescently labeled DNA-streptavidin conjugate was then printed onto each test zone using a piezoelectric microarray printer. Using measured fluorescence intensity (FIG. 1), a molecular density of ˜2.5×10¹³/cm² on each test zone was calculated, which is reasonably consistent with the theoretically predicted value of 8×10¹²/cm² (ρ=1/πr²; r=4 nm, representing the diameter of streptavidin).

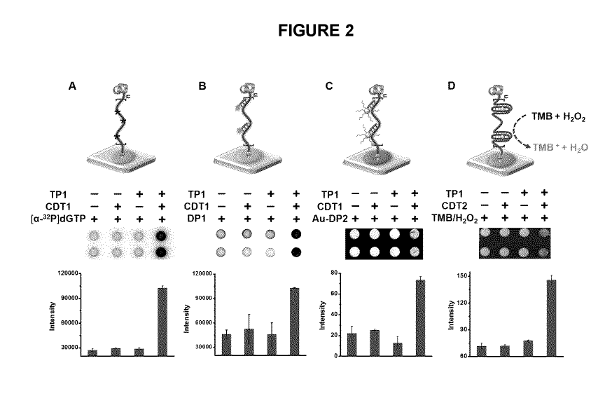

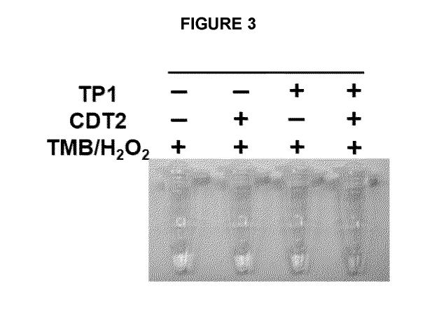

A similar 96-microzone paper device using non-fluorescently labeled primer TP1 was then produced. RCA was performed on paper by placing on a microzone a mixture of circular DNA template (CDT1 or CDT2; see below), phi29 DNA polymerase (ϕ29DP), dNTPs and reaction buffer, followed by incubation at room temperature for 40 min. Four different methods were used to confirm the formation of RCA products (RP) on paper. First, [α-³²P]dGTP was added in the reaction mixture so that the RP became radioactive (FIG. 2A). Second, a fluorophore-labeled DNA probe (DP1) that can hybridize with the RP was used to produce a fluorescence signal (FIG. 2B). Third, gold nanoparticles (AuNPs) functionalized with a complementary DNA probe (DP2) was used to produce a colorimetric signal (FIG. 2C). Note that in these three assays the same circular DNA template CDT1 was used. However, in the final assay, a modified CDT1, named CDT2, was used which was designed to produce a special RP containing repetitive units of a peroxidase-mimicking DNAzyme, PW17, that was able to generate a colorimetric signal (FIG. 2D).^{[10]} Following RCA, the reaction mixture was also taken and placed in a test-tube. No color change was observed (FIG. 3), suggesting that the RP is indeed paper-bound. Taken together, these tests demonstrated that the RCA reaction could be performed on paper printed with a DNA primer.

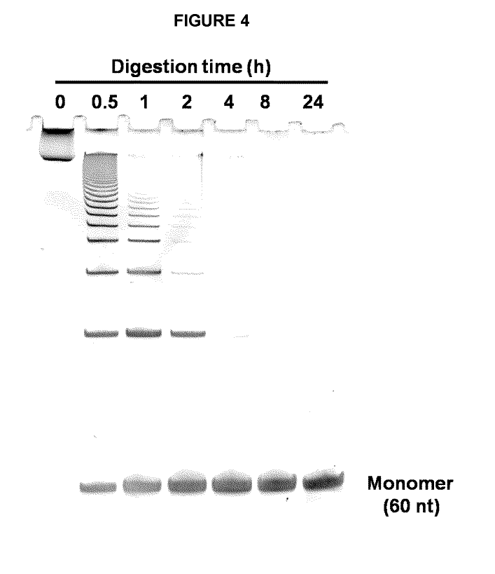

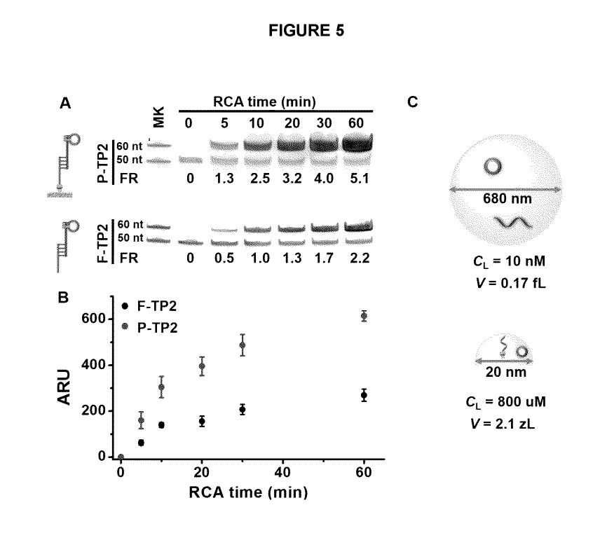

RCA performance on paper relative to solution was investigated next. In order to quantify the long RP on paper, a new DNA primer (TP2) was designed that contained two sequence domains: a 5′ domain that binds to a DNA capture sequence (DC1) printed onto paper microzones and a 3′ domain complementary to the circular template CDT2. After the RCA reaction, urea was added to elute the RP from the paper surface. The recovered RP, which contained a recognition sequence for the restriction enzyme EcoRV, was converted into monomers using EcoRV. Fully digested RP was then analyzed by denaturing polyacrylamide gel electrophoresis (dPAGE). A full monomerization (60 nt) of RP was achieved after a 24-hour digestion (FIG. 4). Through the use of a 50-nt DNA molecule with a defined concentration as an internal control, we were able to determine the fluorescence ratio (FR) of the two bands in each lane (FIG. 5A), and calculate the average repeating units (ARUs) in the RP.

This method was applied to compare the efficiency of solution-based RCA using free TP2 (F-TP2) and solid-phase RCA using paper-bound TP2 (P-TP2). FIG. 5B shows the time-dependent ARU values for the two RCA strategies. It was observed that the overall ARUs of paper-based RCA were much higher than that of solution-based RCA. This result highlights the advantage of paper-based RCA in terms of reaction kinetics, implying that the rate of the enzymatic reaction was faster on paper.

While not wishing to be limited by theory, the efficiency of RCA is dependent on the number and thermal stability (Tₘ) of primer-template duplexes. In general, the Tₘ of a DNA duplex is positively correlated with the localized concentrations of hybridizing DNA strands.^{[11]} For a solution-based RCA system that consists of 10 nM TP and CDT, the volume of a sphere containing one DNA molecule would be 0.17 fL (FIG. 5C), and thus the calculated sphere radius is about 340 nm. For the paper-based RCA strategy, the immobilization process will confine each DNA partner within a hemisphere of less than 10 nm in radius considering its length. Thus the volume of the hemisphere would be 2.1 zL. Under these conditions, the localized concentration of these DNA molecules is estimated to be 800 μM, or 80000-fold higher than in solution. This should increase the estimated Tₘ for the TP-CDT duplex from 48° C. to 67° C. with the increase of effective concentration from 10 nM to 800 μM under the conditions of 100 mM NaCl and 5 mM MgCl₂ at 30° C., making the RCA process more effective on paper.

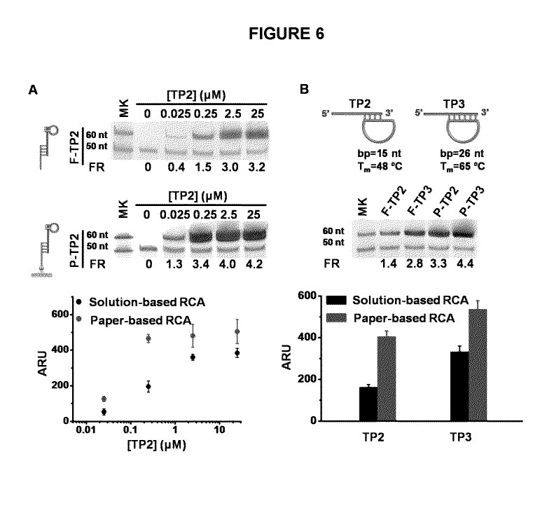

Two additional experiments were conducted to further confirm the increased efficiency of paper-based RCA. The first experiment examined the effect of TP2 concentration on RCA efficiency and the results (FIG. 6A) demonstrate that increasing primer concentration has more profound effect on solution-based RCA than paper-based RCA. The second experiment involved the use of a longer primer, TP3, designed to increase the Tm of primer-template duplex (65° C. for TP3 and 48° C. for TP2). It was found that TP3 significantly boosted the efficiency of solution-based RCA but had a much smaller effect on paper-based RCA (FIG. 6A). These observations confirm the role of immobilization in improving the thermal stability of primer-template duplex, thus facilitating the RCA reaction on paper.

Furthermore, like other DNA replication systems,^{[12]} the rate of RCA is also likely to be affected by the effective concentration of substrates and enzyme.^{[13]} At low substrate concentrations (as in solution), the enzymatic reaction depends on the rate of diffusion of substrate to the enzyme.^{[14]} However, when the effective concentration of substrates (primers, circular templates, dNTPs) reaches saturating levels, as on paper (so-called positive partition effect^{[15]}), the contribution of diffusion should no longer be rate limiting, and thus the system should operate at the intrinsic catalytic rate of the enzyme, significantly enhancing the reaction rate relative to solution.

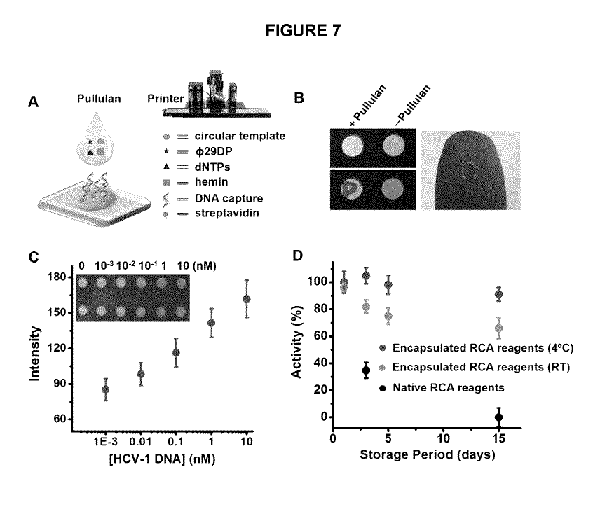

To form a paper-based sensor, paper sensors were combined with the polymeric sugar pullulan to give a simple “all-in-one” POC diagnostic device that can be used in remote settings with a minimal need for special reagent handling and tedious liquid pipetting.^{[16]} The sensors were prepared as follows (FIG. 7A): 1) printing of a DNA capture sequence (DC2) on paper microzones, 2) mixing a pullulan solution with RCA reagents containing a circular DNA template (CDT3), ϕ29DP, dNTPs and hemin, 3) printing the above mixture into the circular test zones as described previously,^{[17]} and 4) air-drying. The transparent pullulan films (letter “P” can be seen) with the encapsulated RCA reagents were obtained on the paper array after drying (FIG. 7B). Addition of a DNA or RNA target leads to the formation of a DC2/CDT3/target complex, which enables the RCA reaction. The RP produced can be detected colorimetrically. The efficiency of the on-paper sample preparation may for example include the DNA extraction method described by Govindarajan et al.^{[18]}

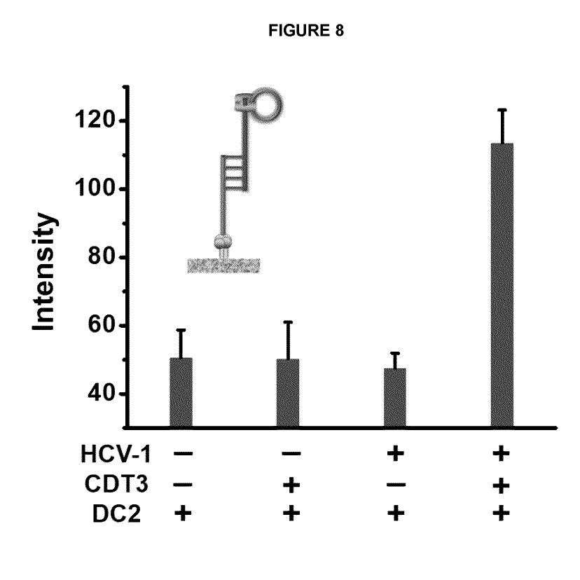

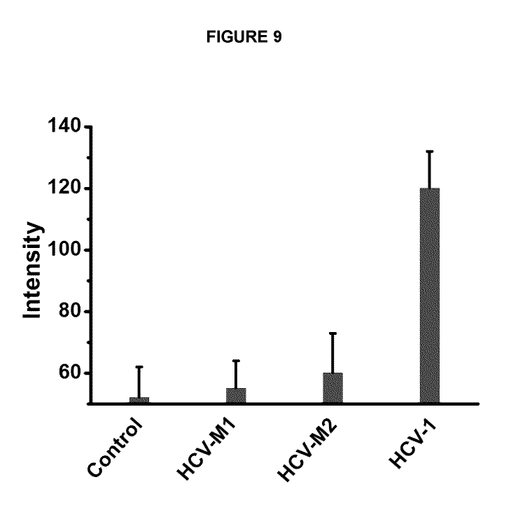

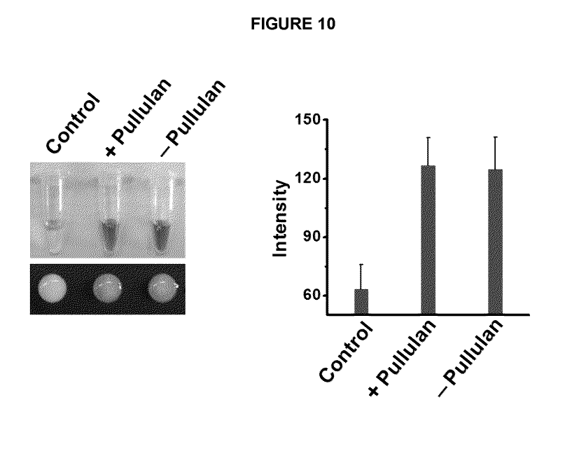

A method of the present application was utilized for the detection of single-stranded HCV-1 DNA (a portion of DNA sequence from the hepatitis C virus genome) to demonstrate analyte-triggered RCA and subsequent detection on paper. As shown in FIG. 7C, RP was observed upon addition of the target, producing colorimetric signals that were proportional to the target concentration, with data calculated using ImageJ. In the absence of HCV-1 DNA, the RCA reaction was not initiated (FIG. 8). The sensor provided a detection limit of 10 pM on the basis of the 36/slope (σ, standard deviation of the blank samples), demonstrating the key advantage of amplification on paper, and showed excellent selectivity against unintended targets HCV-M1 and HCV-M2 with mutations in the HCV-1 sequence (FIG. 9). The positive results further indicate that pullulan does not interfere with the RCA reaction (FIG. 10).

The long-term stability of the “all-in-one” amplification system was next evaluated. As shown in FIG. 7D, RCA reagents stored in solution at room temperature lost 65±6% activity within three days and become completely inactive within 15 days. In contrast, RCA reagents within pullulan films retained 91±5% and 66±8% of their initial activity after storage at 4° C. and room temperature for 15 days, respectively. This result clearly shows that the biomolecules can be effectively protected from thermal denaturation or chemical modification after pullulan encapsulation. This feature is encouraging for room-temperature shipping and storage of the paper-based sensors.

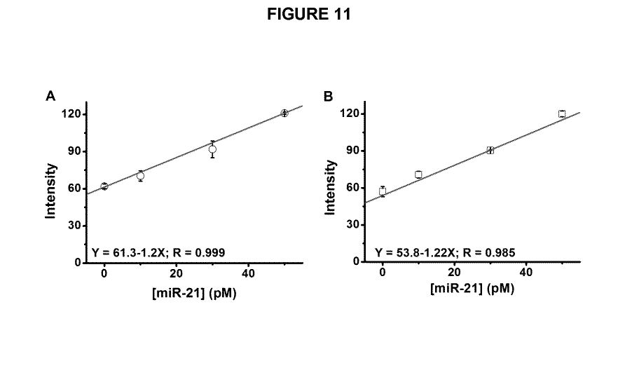

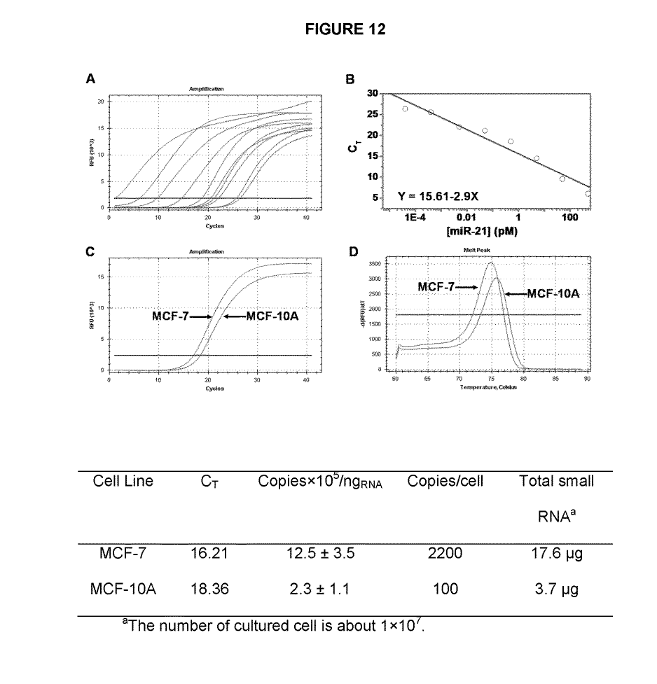

To demonstrate the analytical utility of the paper based RCA system, the sensor was used to detect microRNAs (miRNAs), a group of short (19-25 nucleotides) and endogenous non-protein-coding RNAs, which are promising biomarkers in clinical diagnosis and therapy.^{[19]} The “all-in-one” amplification system was employed to measure the absolute amounts of hsa-miR-21 (miR-21) in enriched small RNA (<200 nt) extracted from human breast cancer cell lines (MCF-7) and normal mammary epithelial cell lines (MCF-10A). Studies have indicated that miR-21 is one of the most abundant miRNAs over-expressed in numerous tumor tissues.^{[20]} The contents of miR-21 in these two cell lines were estimated by the standard addition method and the value of miR-21 was compared with the result of the qRT-PCR method (FIGS. 11 and 12). It was determined that the absolute amount of miR-21 found in MCF-7 and MCF-10A cells were 30.7×10⁵ copies/ng RNA (or 5400 copies/cell) and 6.6×10⁵ copies/ng RNA (or 250 copies/cell), respectively, which is comparable with the values obtained using qRT-PCR (Table 2), thus demonstrating the reliability of our assay.

Overall, the present application demonstrates that the RCA reaction can be performed on a patterned paper device, with the reaction operating with enhanced kinetics, producing rapid and sensitive POC diagnostics. This work also demonstrates that enhanced local reagent concentrations can improve RCA performance relative to solution. The present application further demonstrates that pullulan materials provide both a suitable reagent depot to allow stabilization of labile (bio)reagents and a simple method to immobilize such reagents on paper. The paper sensor with integrated amplification is shown to be suitable for carrying out colorimetric bioassays with minimal steps and without the need for special reagent handling, making this approach particularly suitable for POC testing in resource-limited settings.

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

TABLE 1
Sequences of DNA oligonucleotides

| Name of DNA oligonucleotide | Sequence (5′-3′) |
|---|---|
| Linear precursor of circular | |
| DNA template 1 (CDT1) | |
| LDT1 (60 nt) | ATCTCGACTA CGACTCAGGC TACGGCACGT AGAGCATCAC |
| | CATGATCCTG TGTCTCGGAT |
| Linear precursor of circular | |
| DNA template 2 (CDT2) | |
| LDT2 (45 nt) | ATCTCGACTA AAAACCCAAC CCGCCCTACC CAAAATGTCT |
| | CGGAT |
| Linear precursor of circular | |
| DNA template 3 (CDT3) | |
| LDT3 (45 nt) | ATCTCGACTA TAAAAACCCA ACCCGCCCTA CCCAAAAAAC |
| | GTCGG |
| Linear precursor of circular | |
| DNA template 4 (CDT4) | |
| LDT4 (45 nt) | AGTTCGACTA TAAAAACCCA ACCCGCCCTA CCCAAAATCA |
| | ACATC |
| Circularization DNA templates | |
| CDTa (20 nt) | TAGTCGAGAT ATCCGAGACA |
| CDTb (20 nt) | TATAGTCGAG ATCCGACGTT |
| CDTc (20 nt) | TATAGTCGAA CTGATGTTGA |
| DNA template primers | |
| TP1 (biotinylated at 5′ end) | TAGTCGAGAT ATCCGAGACA |
| TP2 | CTGCCGCTTC CTAACGTTTT TTTTGTCGAG ATATCCGAG |
| TP3 | CTGCCGCTTC CTAACGTTTT TTTTGTCGAG ATATCCGAGA |
| | CACAGGATCA |
| DNA probes | |
| DP1 (FAM labeled at 3′ end) | TGTCTCGGAT ATCTCGACTA |
| DP2 (thiolated at 3′ end) | TGTCTCGGAT ATCTCGACTA |
| DNA capture | |
| DC1 (biotinylated at 3′ end) | CGTTAGGAAG CGGCAG |
| DC2 (biotinylated at 3′ end) | CCGCGTCGCC |
| DC3 (biotinylated at 3′ end) | CTGATAAGCT A |
| DNA template for digestion | |
| DT1 (20 nt) | TGTCTCGGAT ATCTCGACTA |
| Internal control DNA | |
| ICD1 (50 nt) | GACTACGACT CAGGCTACGG CACGTAGAGC ATCACCATGA |
| | TCCTGTGTCT |
| DNA target | |
| HCV-1DNA (21 nt) | GGCGACGCGG GATCCGACGT T |
| HCV-M1DNA (21 nt, HCV-1 with | GCCGATGGGG GATGTTCCGG A |
| mutations) | |
| HCV-M2 DNA (21 nt, HCV-1 with | GTTGACGCGC AAACCTACGT C |
| mutations) | |
| miR-21 | UAGCUUAUCA GACUGAUGUU GA |

TABLE 2
miR-21 amounts (copies/ng RNA × 10⁵) in extracted
total small RNA from MCF-7 and MCF-10A cell lines.

| Cell | Paper-based | | Total small RNA |
|---|---|---|---|
| line^{[a]} | assay | qRT-PCR^{[b]} | (<200 nt, μg) |
| MCF-7 | 30.7 ± 5.2 | 12.5 ± 3.5 | 17.6 |
| MCF- | 6.6 ± 1.3 | 2.3 ± 1.1 | 3.7 |
| 10A | | | |

^{[a]}The number of cultured cell is about 1 × 10⁷.
^{[b]}15 ng of total small RNA per assay. Data are averages ± SD of three independent experiments.

### REFERENCES

[1] a) P. Yager, G. J. Domingo, J. Gerdes, Annu. Rev. Biomed. Eng. 2008, 10, 107-144; b) D. A. Giljohann, C. A. Mirkin, Nature 2009, 462, 461-464; c) M. Urdea, L. A. Penny, S. S. Olmsted, M. Y. Giovanni, P. Kaspar, A. Shepherd, P. Wilson, C. A. Dahl, S. Buchsbaum, G. Moeller, D. C. Hay Burgess, Nature 2006, 444, 73-79; d) P. Yager, T. Edwards, E. Fu, K. Helton, K. Nelson, M. R. Tam, B. H. Weigl, Nature 2006, 442, 412-418; e) L. Kulinsky, Z. Noroozi, M. Madou, Methods Mol. Biol. 2013, 949, 3-23.
[2] a) A. W. Martinez, S. T. Phillips, G. M. Whitesides, Anal. Chem. 2010, 82, 3-10; b) X. Li, D. R. Ballerini, W. Shen, Biomicrofluidics 2012, 6, 011301; c) A. K. Yetisen, M. S. Akram, C. R. Lowe, Lab Chip 2013, 13, 2210-2251; d) C. Parolo, A. Merkoçi, Chem. Soc. Rev. 2013, 42, 450-457; e) D. D. Liana, B. Raguse, J. J. Gooding, E. Chow, Sensors 2012, 12, 11505-11526.
[3] S. Su, M. M. Ali, C. D. Filipe, Y. Li, R. Pelton, Biomacromolecules 2008, 9, 935-941.
[4] E. Carrilho, A. W. Martinez, G. M. Whitesides, Anal. Chem. 2009, 81, 7091-7095.
[5] A. Savolainen, Y. Zhang, D. Rochefort, U. Holopainen, T. Erho, J. Virtanen, M. Smolander, Biomacromolecules 2011, 12, 2008-2015.
[6] a) C. M. Cheng, A. W. Martinez, J. Gong, C. R. Mace, S. T. Phillips, E. Carrilho, K. A. Mirica, G. M. Whitesides, Angew. Chem., Int. Ed. 2010, 49, 4771-4774; b) A. W. Martinez, S. T. Phillips, G. M. Whitesides, Proc. Natl. Acad. Sci. USA 2008, 105, 19606-19611; c) H. Liu, Y. Xiang, Y. Lu, R. M. Crooks, Angew. Chem. Int. Ed. 2012, 51, 6925-6928; d) S. M. Z. Hossain, C. Ozimok, C. Sicard, S. D. Aguirre, M. M. Ali, Y. Li, J. D. Brennan, Anal. Bioanal. Chem. 2012, 403, 1567-1576; e) A. W. Martinez, S. T. Phillips, M. J. Butte, G. M. Whitesides, Angew. Chem. 2007, 119, 1340-1342; Angew. Chem. Int. Ed. 2007, 46, 1318-1320.
[7] a) J. Li, J. Macdonald, Biosens. Bioelectron. 2015, 64, 196-211; b) K. P. F. Janssen, K. Knez, D. Spasic, J. Lammertyn, Sensors 2013, 13, 1353-1384; c) L. Yan, J. Zhou, Y. Zheng, A. S. Gamson, B. T. Roembke, S. Nakayama, H. O. Sintim, Mol. BioSyst. 2014, 10, 970-1003.
[8] a) C. Jung, A. D. Ellington, Acc. Chem. Res. 2014, 47, 1825-1835; b) L. M. Zanoli, G. Spoto, Biosensors 2013, 3, 18-43; c) A. Niemz, T. M. Ferguson, D. S. Boyle, Trends Biotechnol. 2011, 29, 240-250; d) P. Craw, W. Balachandran, Lab Chip 2012, 12, 2469-2486; e) P. J. Asiello, A. J. Baeumner, Lab Chip 2011, 11, 1420-1430.
[9] a) A. Fire, S. Q. Xu, Proc. Natl. Acad. Sci. USA 1995, 92, 4641-4645; b) D. Liu, S. L. Daubendiek, M. A. Zillman, K. Ryan, E. T. Kool, J. Am. Chem. Soc. 1996, 118, 1587-1594; c) W. Zhao, M. M. Ali, M. A. Brook, Y. Li, Angew. Chem. Int. Ed. 2008, 47, 6330-6337. d) M. M. Ali, F. Li, Z. Zhang, K. Zhang, D. K. Kang, J. A. Ankrum, X. C. Le, W. Zhao, Chem. Soc. Rev. 2014, 43, 3324-3341.
[10] a) P. Travascio, Y. Li, D. Sen, Chem. Biol. 1998, 5, 505-517; b) H. Z. He, D. S. Chan, C. H. Leung, D. L. Ma, Nucleic Acids Res. 2013, 41, 4345-4359; c) Z. Cheglakov, Y. Weizmann, B. Basnar, I. Willner, Org. Biomol. Chem. 2007, 5, 223-225.
[11] Q. Zhang, F. Li, B. Dever, C. Wang, X. F. Li, X. C. Le, Angew. Chem. Int. Ed. 2013, 52, 10698-10705.
[12] a) J. Ninio, Proc. Natl. Acad. Sci. USA 1987, 84, 663-667; b) M. Salas, Annu. Rev. Biochem. 1991, 60, 39-71.
[13] a) L. Blanco, A. Bernad, J. M. Lharo, G. Martin, C. Garmendia, M. Salas, J. Biol. Chem. 1989, 264, 8935-8940; b) L. Blanco, J. M. Lazaro, M. D. Vega, A. Bonnin, M. Salas, Proc. Natl. Acad. Sci. USA. 1994, 91, 12198-12202.
[14] a) P. B. Gaspers, C. R. Robertson, A. P. Gast, Langmuir 1994, 10, 2699-2704; b) P. B. Gaspers, A. P. Gast, C. R. Robertson, J. Colloid Interface Sci. 1995, 172, 518-529; c) G. Trigiante, A. P. Gast, C. R. Robertson, J. Colloid Interface Sci. 1999, 213, 81-86.
[15] R. C. Rodrigues, C. Ortiz, A. Berenguer-Murcia, R. Torres, R. Fernandez-Lafuente, Chem. Soc. Rev. 2013, 42, 6290-6307.
[16] S. Jahanshahi-Anbuhi, K. Pennings, V. Leung, M. Liu, C. Carrasquilla, B. Kannan, Y. Li, R. Pelton, J. D. Brennan, C. D. Filipe, Angew. Chem. Int. Ed. 2014, 53, 6155-6158.
[17] B. Kannan, S. Jahanshahi-Anbuhi, R. H. Pelton, Y. Li, C. D. M Filipe, J. D. Brennan, Anal. Chem. 2015, 87, 9288-9293.
[18] A. V. Govindarajan, S. Ramachandran, G. D. Vigil, P. Yager, K. F. BOhringer, Lab Chip 2012, 12, 174-181.
[19] a) L. He, G. J. Hannon, Nat. Rev. Genet. 2004, 5, 522-531; b) S. L. Ameres, P. D. Zamore, Nat. Rev. Mol. Cell Biol. 2013, 14, 475-488; c) B. M. Ryan, A. I. Robles, C. C. Harris, Nat. Rev. Cancer 2010, 10, 389-402; d) H. Dong, J. Lei, L. Ding, Y. Wen, H. Ju, X. Zhang, Chem. Rev. 2013, 113, 6207-6233; e) J. Li, S. Tan, R. Kooger, C. Zhang, Y. Zhang, Chem. Soc. Rev. 2014, 43, 506-517.
[20] A. M. Krichevsky, G. Gabriely, J. Cell. Mol. Med. 2009, 13, 39-53.

1. A nucleic acid sensor probe for detection of target nucleic acid comprising:
a) a capture oligonucleotide that comprises a nucleic acid sequence that is complementary to a first nucleic acid sequence of the target oligonucleotide;
b) a solid support immobilizing the capture oligonucleotide; and
c) reagents for performing rolling-circle amplification (RCA) of the target oligonucleotide.

2. The nucleic acid sensor probe of claim 1, wherein the reagents for performing RCA are comprised in a stabilized composition.

3. The nucleic acid sensor probe of claim 2, wherein the reagents are encapsulated in a stabilizing matrix or are freeze dried.

4. The nucleic acid sensor probe of claim 3, wherein the reagents for performing RCA are encapsulated with pullulan.

5. The nucleic acid sensor probe of claim 1, wherein the capture oligonucleotide is biotinylated and bound with streptavidin on a membrane surface.

6. The nucleic acid sensor probe of claim 1, wherein the solid support is paper or a paper-based material.

7. The nucleic acid sensor probe of claim 1, wherein the reagents for performing RCA comprise a circular template comprising a nucleic acid sequence that is complementary to a second nucleic acid sequence of the target oligonucleotide and a nucleic acid polymerase having exonuclease activity.

8. The nucleic acid sensor probe of claim 7, wherein the nucleic acid polymerase is a DNA polymerase having 3′ to 5′ exonuclease activity or an RNA polymerase having 3′ to 5′ exonuclease activity.

9. The nucleic acid sensor probe of claim 8, wherein the nucleic acid polymerase is a DNA polymerase.

10. (canceled)

11. The nucleic acid sensor probe of claim 1, further comprising one or more reporter molecules for detection of the target oligonucleotide.

12. The nucleic acid sensor probe of claim 11, wherein the one or more reporter molecules for detection of the target oligonucleotide comprise a detection system selected from a fluorescent system, a colorimetric system and an electrochemical system.

13. The nucleic acid sensor probe of claim 1, wherein the capture oligonucleotide and/or target nucleic acid are selected from DNA molecules and RNA molecules.

14. (canceled)

15. (canceled)

16. The nucleic acid sensor probe of claim 1, wherein the capture oligonucleotide and the encapsulated reagents are absorbed onto the solid support by non-chemical means.

17. The nucleic acid sensor probe of claim 16, wherein the non-chemical means is printing selected from inkjet printing, wax printing or screen printing technology.

18. (cancelled)

19. A method of determining the presence of target nucleic acid in a sample comprising:
a) exposing the nucleic acid sensor probe of claim 1 to the sample;
b) performing rolling-circle amplification (RCA) on the nucleic acid sensor probe under conditions to generate single-stranded nucleic acid molecules; and
c) detecting single-stranded nucleic acid molecules generated in b),
wherein the detection of the single-stranded nucleic molecule in c) indicates the presence of the target nucleic acid in the sample.

20. The method of claim 19, wherein the detection of single-stranded nucleic acid molecules generated by RCA is indicated by a colorimetric system, a fluorescence system, a radiolabeling system or an electrochemical system.

21. The method of claim 19, wherein the sample is from a microorganism.

22. The method of claim 19, wherein the sample is a biological sample, and the presence of the target nucleic acid in the sample is indicative of, or associated, with a disease, disorder or condition.

23. The method of claim 19, wherein the single-stranded nucleic acid molecule is a single-stranded DNA molecule or a single-stranded RNA molecule.

24. A target oligonucleotide detection kit comprising a nucleic acid sensor probe of claim 1.
